(19) United States
(12) Patent Application Publication
(10) Pub. No.: US 2023/0248717 A1
(43) Pub. Date: Aug. 10, 2023

(54) N-(3-HYDROXYPYRIDINE-2-CARBONYL)GLYCINE-BASED ANTITUMOR DRUG SENSITIZER AND APPLICATION THEREOF

(71) Applicant: ZHEJIANG UNIVERSITY, Hangzhou (CN)

(72) Inventors: Youqing SHEN, Hangzhou (CN); Jing LIU, Hangzhou (CN); Zhihao ZHAO, Hangzhou (CN)

(73) Assignee: ZHEJIANG UNIVERSITY, Hangzhou (CN)

(21) Appl. No.: 18/012,999

(22) PCT Filed: Oct. 29, 2020
(86) PCT No.: PCT/CN2020/124755

§ 371 Date: Dec. 27, 2022

(30) Foreign Application Priority Data
Dec. 17, 2019 (CN) 201911306631.5

(51) Int. Cl. A61K 31/47; A61K 31/4439; A61K 31/4418; A61K 31/4412; A61K 9/107

(52) U.S. Cl. CPC A61K 31/47; A61K 31/4439; A61K 31/4418; A61K 31/4412; A61K 9/107

(57) ABSTRACT

An N-(3-hydroxypyridine-2-carbonyl)glycine-based antitumor drug sensitizer and an application thereof are provided. In the present disclosure, a compound of formula (I) can down-regulate PD-L1 of tumor cells, promote polarization of macrophages from M2 to M1, inhibit an expression of indoleamine 2,3-dioxygenase, and improve an immunotherapy curative effect. The compound can also reduce the expression of hypoxia-inducible factor-1α in tumor cells, down-regulate an expression of P-glycoprotein, enhance the killing effect of a chemotherapeutic drug on tumor cells, and enhance immunogenic cell death. The compound of formula (1) has a significantly improved antitumor effect after being used in combination with a chemotherapeutic drug and shows a desirable prospect for use.

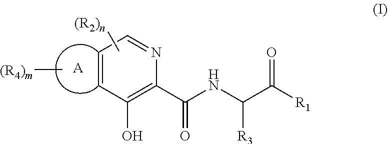

### CROSS REFERENCE TO THE RELATED APPLICATIONS

This application is the national phase entry of International Application No. PCT/CN2020/124755, filed on Oct. 29, 2020, which is based upon and claims priority to Chinese Patent Application No. 201911306631.5, filed on Dec. 17, 2019, the entire contents of which are incorporated herein by reference.

### TECHNICAL FIELD

The present disclosure relates to the technical field of medicine, in particular, to the use of N-(3-hydroxypyridine-2-carbonyl)glycine and derivatives thereof in the preparation of an antitumor drug sensitizer.

### BACKGROUND

With the in-depth study of tumor immunology, researchers have foun.d that tumors, especially solid tumors, usually developed into a complex tissue structure composed of cancer cells, fibroblasts, lymphocytes, blood vessels, and various extracellular matrices to achieve rapid growth. This complex tissue structure is also called a tumor microenvironment (TME). Compared with normal tissues, tumor cells generally have lots of unique microenvironmental characteristics, such as relatively low pH, increased interstitial pressure, vascular aberration, insufficient oxygen supply, and increased reactive oxygen species (ROS) (Microenvironmental regulation of tumor progression and metastasis, Nature Medicine, 2013, 11 (19): 1423; The immunosuppressive tumor network: myeloid-derived suppressor cells, regulatory T cells and natural killer T cells, Immunology 2013, 2 (138): 105). Tumor cells have a more vigorous metabolism than normal cells, and the abnormality and uneven distribution of blood vessels in tumor tissues prevent oxygen delivery, resulting in a large number of hypoxic areas in the tumor (Hypoxia-inducible factor 1 is a basic-helix-loop-helix-PAS heteroditner regulated by cellular 02 tension, Proceedings of The National Academy of Science of The United States of America, 1995, 92 (12): 5510; The tumor suppressor protein VEIL targets hypoxia-inducible factors for oxygen-dependent proteolysis, Nature, 1999, 6733 (399): 271).

Numerous studies have shown that hypoxia of tumor tissues is closely related to the tumor immunosuppressive microenvironment. For example, tumor hypoxia leads to the generation of M2-type tumor-associated macrophages (TAMs), thus suppressing tumor immune responses (HIF-1 alpha is essential for myeloid cell-mediated inflammation, Cell. 2003, 112 (5): 645-657; Hypoxian cancer: significance and impact on clinical outcome, Cancer and Metastasis Reviews, 2007, 2 (26): 225).

Tumor hypoxia can inhibit the proliferation and activation of tumor-infiltrating lymphocytes (TILs), especially cytotoxic lymphocytes (CTLs), thereby affecting the function of effector T cells (Obesity in C57BL/6J mice is characterized by adipose tissue hypoxia and cytotoxic I-cell infiltration, International Journal of Obesity, 2008, 3 (32): 451; Inhibitory effect of tumor cell-derived lactic acid on human T cells, Blood, 2007, 9 (109):3812; HIE Transcription Factors, Inflammation, and. Immunity, Immunity, 2014, 4 (41):518).

Tumor hypoxia also induces the overexpression of PD-L1 in cancer cells and macrophages, which in turn induces programmed cell death of CTL cells (A Mechanism of hypoxia-mediated escape from adaptive immunity in cancer cells, Cancer Research, 2014, 3 (74): 665; Genomic correlates of response to immune checkpoint therapies in clear cell renal cell carcinoma, Science, 2018, 359 (6377): 801),

In addition, the unique tumor microenvironment also attracts a large number of immunosuppressive cells, including regulatory T cells and myeloid-derived suppressor cells (MDSCs). These cells secrete some immunosuppressive cytokines, such as indoleamine 2,3-dioxygenase (IDO), hydrogen peroxide, peroxynitrite, and other reactive oxygen/nitrogen free radicals. Such cytokines may further inhibit the antigen-presenting effect of antigen-presenting cells, thereby inhibiting the infiltration, proliferation, and differentiation of T lymphocytes in tumors. This leads to the failure of antitumor immunotherapy and promotes tumor growth and metastasis (Tumor microenvironment complexity: emerging roles in cancer therapy, Cancer Research, 2012, 72 (10): 2473; Myeloid-derived suppressor cells: Critical cells driving immune suppression in the tumor microenvironment, Immunotherapy of Cancer, 2015, 128: 95; Targeting the tumor microenvironment: removing obstruction to anticancer immune responses and immunotherapy, Annals of Oncology, 2016, 27(8): 1482). Therefore, the immunosuppressive microenvironment in solid tumors is different from that in normal tissues, which enables tumor cells to escape the attack of the immune system. This is one of the key factors limiting the efficacy of tumor immunotherapy.

Hypoxic heterogeneity also increases the drug resistance of solid tumors. When the tumor has a volume exceeding 3 mm3; the tumor shows a hypoxic state inside (Intratumoral hypoxia; radiation resistance, and HIF-1, Cancer Cell, 2004, 5 (5):405). Solid tumor cells are less sensitive to chemotherapeutic drugs in the hypoxic environment, such that hypoxia is an important factor of chemoresistance (Molecular targeting therapy of cancer: drug resistance, apoptosis and survival signal, Cancer Science, 2003, 94 (1): 15; Hypoxia-inducible factor-1a contributes to hypoxia-induced chemoresistance in gastric cancer, Cancer Science, 2008, 99 (1): 121). In addition, chemotherapeutic drug treatment may further remodel the immune microenvironment of the tumor, such as up-regulating the expression of PD-L1, IDO, or HIF-1α, such that the tumor is protected from autoimmunity, thus further reducing the efficacy.

The immunosuppressive agents can reverse the immune microenvironment, such as blocking the inhibition of T cells by tumor cells with PD-1/PD-L1 antibodies, which allows T cells to restore the function of recognizing and eliminating tumor cells. This is the current general scheme of tumor immunotherapy, showing high efficacy on target-matched tumors (The blockade of immune checkpoints in cancer immunotherapy, Nature Reviews Cancer, 2012, 4 (12): 252; Safety, activity, and immune correlates of anti-PD-1 antibody in cancer. New England Journal of Medicine, 2012, 26 (366): 2443). In addition, chemotherapeutic drugs can stimulate tumor cells to produce immunogenic death, induce the expression of signals such as calreticulin, and attract T cells and dendritic cells into the tumor, thereby enhancing the antitumor immune response (Immunobiology of dendritic cells. Annual Review of Immunology, 2000, 18: 767; Cancer immunoediting: integrating immunity's roles in cancer suppression and promotion, Science. 2011, 331 (6024): 1565).

Therefore, the combination of certain chemotherapeutic drugs with PD-1/PD-L1 antibody can enhance the efficacy (Adoptive immunotherapy for cancer: harnessing the T cell response, Nature Reviews Immunology, 2012, 12 (4): 269; Nishikawa H et al., Regulatory T cells in cancer immunotherapy. Current Opinion In immunology, 2014, 27: 1; Dendritic cells as mediators of tumor-induced tolerance in metastatic melanoma. International Journal of Cancer. 1997, 73 (3): 309). However, PD-1/PD-L1 antibodies have a high risk of side effects, inconvenient preparation and storage, and a high cost.

N-(4-Hydroxy-1-methyl-7-phenoxyisoquinoline-3-carbonyl)glycine Roxadustat, ROX) by mimicking ketoglutaric acid, one of the substrates of prolyl hydroxylase (PH), can inhibit the prolyl hydroxylase of hypoxia-inducible factors. Therefore, Roxadustat maintains or even increases a level of the hypoxia-inducible factor in normal cells, thereby increasing the expression level of erythropoietin, and increasing the expression levels of erythropoietin receptors and proteins that promote iron absorption and circulation. Accordingly, Roxadustat is clinically used as a drug for the treatment of renal anemia.

### SUMMARY

The present disclosure provides a small-molecule antitumor drug sensitizer based on N-(3-hydroxypyridine-2-carbonyl)glycine and derivatives thereof to improve the effects of immunization and chemotherapy on tumors.

The disclosure adopts the following technical solutions to solve the above technical problems:

The present disclosure provides an antitumor drug sensitizer being a compound of formula (I)

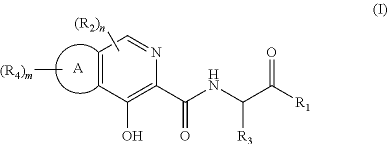

or a pharmaceutically acceptable salt of the compound, vkbere

R₁ is selected from the group consisting of H, OH, NH₂, C₁₋₂₀ alkyl, —O—C₁₋₂₀ alkyl, —NR—C₁₋₂₀ alkyl, and —O—C₆₋₁₂ aryl;

R₂ is selected from the group consisting of H, F, Cl, Br, I, OH, NH₂, NO₂, CN, C₁₋₂₀ alkyl, —O—C₁₋₂₀ alkyl, —NH13 C₁₋₂₀ alkyl, C₆₋₁₂ aryl, —O—C₆₋₁₂ aryl, and 5- to 10-membered heteroaryl; the C₁₋₂₀ alkyl, the —O—C₁₋₂₀ alkyl, the —NH—Cr₁₋₂₀ alkyl, the C₆₋₁₂ aryl, the —O—C₆₋₁₂ aryl, and the 5- to 10-membered heteroaryl are optionally substituted by 1, 2, or 3 Ra;

R₃ is selected from the group consisting of Ft, F, Cl, Br, I, OH, NH₂, NO₂, C_{1.20} alkyl, —O—C₁₋₂₀ alkyl, —NH—C₁₋₂₀ alkyl, C₆₋₁₂ aryl, and —O—C₆₋₁₂ aryl;

R₄ is selected from the group consisting of H, F, Cl, Br, I, OH, NH₂, NO2, CN, C₁₋₂₀ alkyl, —O—C₁₋₂₀ alkyl, —NH—C₁₋₂₀ alkyl, C₆₋₁₂ aryl, —O—C₆₋₁₂ aryl, and 5- to 10-membered heteroaryl; the C₁₋₂₀ alkyl, the —O—C₁₋₂₀ alkyl, the —NH—C₁₋₂₀ alkyl, the C₆-I2 aryl, the —O—C₆₋₁₂ aryl, and the 5- to 10-membered heteroaryl are optionally substituted by 1, 2, or 3 R_{b};

ring A is phenyl or absent;

Rₐ is independently selected from the group consisting of F, Cl, Br, I, OH, NO₂, CN, C₁₋₃ alkyl, and C₁₋₃alkylphenyl; and the C₁₋₃ alkyl or the C₁₋₃alkylphenyl is optionally substituted by 1, 2, or 3 halogens;

R_{b} is independently selected from the group consisting of F, Cl, Br, I, OH, NH₂, NO₂, and CN;

m is 0, 1, 3, or 4; and

n is 0, 1, or 2.

The compound of formula (I) inhibits the expression of prolyl hydroxylase 3 (PHD₃) under hypoxia, then down-regulates the expression of pyruvate kinase M2 (PKM2), and finally inhibits the expression of HIF-1α. As the expression of HIF-1α is inhibited, a dimerization degree of HIF-1α and HIT-1F is also greatly reduced, such that the expression of downstream factors such as PD-L1 and P-gp is also reduced.

Effects of the compound of formula (I) in tumor immunotherapy include reducing the expression of PD-L1 and CD47 in tumor cells, enhancing the activity of T lymphocytes and infiltrating the tumor tissue, inhibiting the expression of indoleamine 2,3-dioxygenase and increasing the activity of cytotoxic T cells, promoting the transformation of macrophages from M2 to M1, reversing the immunosuppressive state, and promoting the immunogenic death of tumor cells, thereby enhancing the body's immune response to tumors to enhance the tumor immunotherapy.

Effects of the compound of formula (I) in chemotherapy sensitization include reducing the expressions of a and multidrug resistance protein P-gp in tumor cells, inhibiting the multidrug resistance of tumors, promoting the endocytosis of chemotherapeutic drugs in tumor cells, and increasing the sensitivity of tumor cells to chemotherapeutic drugs, thereby increasing an antitumor effect of the chemotherapeutic drugs.

Further, the compound of formula (1) is selected from the group consisting of:

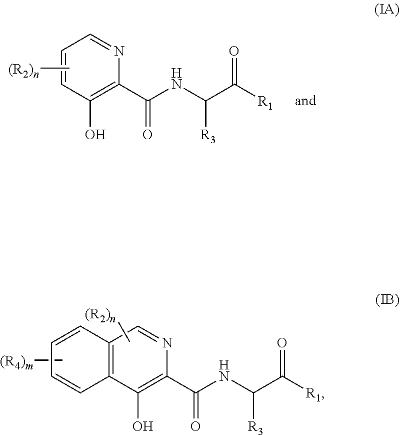

where

R₁, R₂, R₃, R₄, n, and m is as defined in the present disclosure,

Further, Rₐ is selected from the group consisting of F, Cl, Br, I, OH, NH₂, NO₂, CN, and

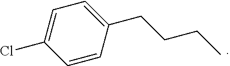

Further, R₂ is selected from the group consisting of F, Cl, Br, I, OH, NH₂, NO₂, CN, C₁₋₃ alkyl, —O—C₁₋₃ alkyl, alkyl, phenyl, —O-phenyl or —O-pyrazolyl, pyrrolyl, pyrazolyl, and triazolyl; the C₁₋₃ alkyl, the —O—C₁₋₃ alkyl, the —NH—C₁₋₃ alkyl, the phenyl, the —O-phenyl or —O-pyrazolyl, the pyrrolyl, the pyrazolyl, and the triazolyl are optionally substituted by 1, 2, or 3 Rₐ.

Further, R₂ is selected from

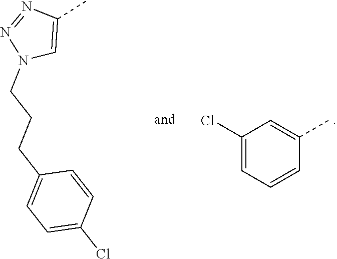

Further, R₃ is selected from the group consisting of H, F, Cl, Br, I, OH, NH₂, NO₂, and CN.

Further, R₄ is selected from the group consisting of H, F, Cl, Br, I, OH, NH₂, NO₂, CN, and

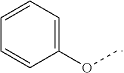

Further, the compound of formula (I) is selected from the group consisting of:

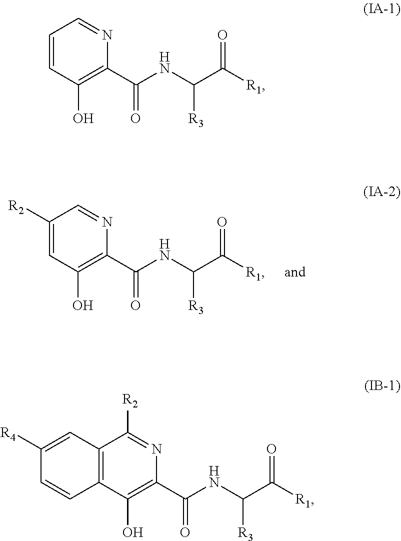

R₁, R₂, R₃, and R₄ are as defined in the present disclosure. Further, the antitumor drug sensitizer is a compound of the following formula (1), (2), (3), (4), (5), (6), (7); or (8):

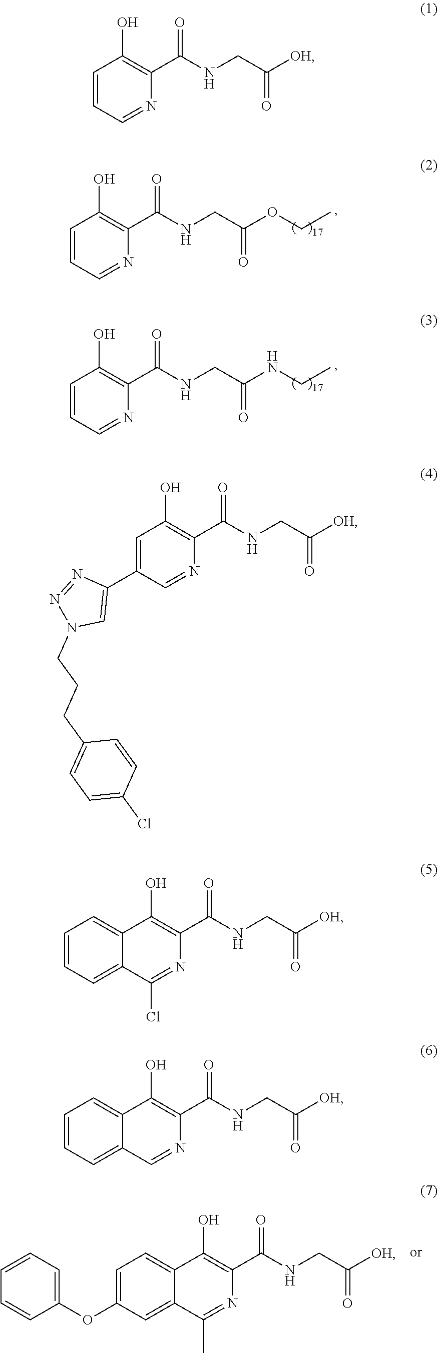

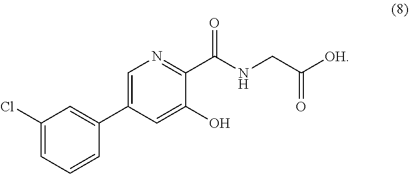 [SMILES: O=C(O)CNC(=O)c1ncc(-c2cccc(Cl)c2)cc1O]

Experiments have found that the antitumor drug sensitizer can reduce the dimerization degree of HIF-1α and HIF-1β by down-regulating the expression of HIF-1α, thereby down-regulating a series of downstream factors, such as PD-L1, CD47 and P-gp.

Compared with other compounds, compound 7 not only retains the carboxyl moiety (while compounds 2 or 3 need to be hydrolyzed to expose the active carboxyl group) but also is more hydrophobic than compounds 1, 5, and 6, which can be easily taken up by tumor cells and thus has a better therapeutic effect.

In the present disclosure, the antitumor drug sensitizer can be used in combination with an antitumor drug in different proportions.

The antitumor drug sensitizer and the antitumor drug have a mass ratio of (0.1-20):1.

The antitumor drug includes cyclophosphamide, 5-fluorouracil, raltitrexed, doxorubicins, cytidines, antifolates, paclitaxels, gemcitabine, platinum drugs, camptothecin and derivatives thereof, tripterine, vincristines, gambogic acid, or molecular targeted drugs.

In the present disclosure, the tumor is malignant; the malignant tumor includes hematological cancer, gastric cancer, esophageal cancer, colorectal cancer, breast cancer, melanoma, brain cancer, pancreatic cancer, lung cancer, bladder cancer, ovarian cancer, liver cancer, or bile duct cancer.

The present disclosure further provides a pharmaceutical composition, including a therapeutically effective amount of the antitumor drug sensitizer and a pharmaceutically acceptable carrier.

The pharmaceutically acceptable carrier is selected from the group consisting of water, liposomes, polymeric micelles, and inorganic nanocarriers.

The antitumor drug sensitizer has a long blood circulation time after being prepared into a nano-formulation and can accumulate more effectively in tumor tissues through an enhanced permeability and retention effect of the tumor, thereby further improving the antitumor effect.

In the present disclosure, the pharmaceutical composition can be directly administered by conventional oral administration, injection, and the like.

The present disclosure further provides a preparation method for a liposome carrying the antitumor drug sensitizer, including the following steps:

preparation of a liposome membrane: dissolving phospholipid or PEGylated phospholipid or a mixture thereof with the antitumor drug sensitizer in a solvent I and concentrating at 4° C. to 60° C. to form a membrane and

hydration: adding deionized water or a buffer solution I with an appropriate pH value to the membrane, hydrating at 4° C. to 60° C. for 12 h to 48 h, and performing dialysis on an obtained hydration product in a dialysis bag at room temperature for 6 h to 48 h.

The phospholipid is selected from the group consisting of phosphatidylcholine, phosphatidylethanolamine, dioleoylphosphatidylethanolamine, cholesterol hemisuccinate, and distearatephosphatidylethanolamine. The PEGylated phosphol ipid includes phosphatidylethanolamine-polyethylene glycol, and the polyethylene glycol has a molecular weight of 2,000 to 10,000.

Buffer solution 1 has an appropriate pH value of 2 to 9.

Buffer solution 1 with an appropriate pH is PBS.

Solvent 1 is selected from the group consisting of dichloromethane (DCM), chloroform, methanol, and a mixture thereof.

Solvent 1 is a mixture of chloroform and methanol in a volume ratio of (1-8):1.

The dialysis bag has a molecular weight of 500 KD to 10,000 KD.

The present disclosure further provides a preparation method for a micellar composition loaded with the antitumor drug sensitizer, including the following steps:

preparation of a micellar membrane: dissolving a polymer and the antitumor drug sensitizer in a solvent 2 and concentrating at 4° C. to 60° C. to form a membrane and

hydration: adding deionized water or a buffer solution 2 with an appropriate pH value to the membrane, hydrating at 4° C. to 60° C. for 2 h to 48 h, and passing through a filter membrane.

The copolymer is selected from the group consisting of a polyethylene glycol-polylactide block copolymer and a polyoxyethylene-polyoxypropylene ether block copolymer.

Solvent 2 is selected from the group consisting of DCM, trichloromethane, tetrahydrofuran, acetonitrile, and acetone.

The filter membrane has a pore size of 150 nm to 250 nm.

The filter membrane has a pore size of 200 nm.

The buffer solution 2 has an appropriate pH value of 2 to 9.

The buffer solution 2 with an appropriate pH is PBS.

The present disclosure has the following beneficial effects:

In the present disclosure, the compound of formula (I) can reverse the multidrug resistance of tumors by simultaneously inhibiting HIF-1α and P-gp, inhibiting the expression of immunosuppressive molecules such as PD-L1 and CD47, and enhancing an immune response of the body to tumors, thereby enhancing chemotherapy and immunotherapy effects of the tumor. Compared with the immune checkpoint inhibitor PD-1/PD-L1 antibodies, the N-(3-hydroxypyridine-2-carbonyl)glycine and derivatives thereof are small-molecule compounds with a defined structure, which can be easily synthesized.

Definition and Description of Terms

Unless otherwise specified, the following terms and phrases used herein are intended to have the following meanings. A particular term or phrase when not specifically defined should not be considered indeterminate or unclear but should be understood in its ordinary meaning. When a trade name appears herein, it is intended to refer to its corresponding commercial product or its active ingredient. As used herein, the term “pharmaceutically acceptable” refers to those compounds, materials, and/or compositions in their dosage forms that are determined within the scope of sound medical judgment and are suitable for use in contact with human and animal tissues without undue toxicity, irritation, allergic reactions, or other problems or complications commensurate with a reasonable benefit/risk ratio.

The term “pharmaceutically acceptable salt” refers to salts of the compounds in the present disclosure, which are prepared from compounds with specific substituents discovered by the present disclosure, such as relatively non-toxic acids or bases. When the compounds of the present disclosure include relatively-acidic functional groups, base addition salts can be obtained by contacting the neutral forms of such compounds with a sufficient amount of base in a pure solution or a suitable inert solvent. Pharmaceutically acceptable base addition salts include sodium, potassium, calcium, or ammonium salts; organic amine salts; magnesium salts; or similar salts. When the compounds of the present disclosure include relatively-alkaline functional groups, acid addition salts can be obtained by contacting the neutral forms of such compounds with a sufficient amount of acid in a pure solution or a suitable inert solvent. Pharmaceutically acceptable acid addition salts include inorganic acid salts; the inorganic acids include hydrochloric acid, hydrobromic acid, nitric acid, carbonic acid, bicarbonate, phosphoric acid, monohydrogen phosphate, dihydrogen phosphate, sulfuric acid, hydrogen sulfate, hydroiodic acid, and phosphorous acid. The pharmaceutically acceptable acid addition salts also include organic acid salts; the organic acids include acetic acid, propionic acid, isobutyric acid, maleic acid, malonic acid, benzoic acid, succinic acid, suberic acid, fumaric acid, lactic acid, mandelic acid, phthalic acid, benzenesulfonic acid, p-toluenesulfonic acid, citric acid, tartaric acid, and methanesulfonic acid. The pharmaceutically acceptable acid addition salts also include salts of amino acids such as arginine and the like, and salts of organic acids such as glucuronic acid. Certain specific compounds of the present disclosure contain both basic and acidic functional groups and thus can be converted into either base or acid addition salts.

In the present disclosure, the pharmaceutically acceptable salt can be synthesized from a parent compound containing acid radicals or basic groups by conventional chemical methods. In general, such salts are prepared by conducting a reaction on these compounds in a free acid or base form with a stoichiometric amount of the appropriate base or acid in water, an organic solvent, or a mixture thereof.

In the present disclosure, in addition to the salt form, the compound also has a prodrug form. Prodrugs of the compounds described herein are readily chemically altered under physiological conditions to be converted to the compounds of the present disclosure. Furthermore, prodrugs can be converted to the compounds of the present disclosure by chemical or biochemical methods in an in vivo environment.

In the present disclosure, certain compounds may exist in unsolvated or solvated forms, including hydrated forms. Generally, the solvated and unsolvated forms are equivalent and are intended to be included within the scope of the present disclosure.

In the present disclosure, the compounds may exist in specific geometric or stereoisomeric forms. It is envisaged that all such compounds include cis- and trans-isomers, (−)- and (+)-enantiomers, (R)- and (S)-enantiomers, diastereomers. (D)-isomer, (L)-isomer, and racemic and other mixtures thereof. For example, there are mixtures enriched by enantiomers or diastereomers, all of which are within the scope of the present disclosure. Additional asymmetric carbon atoms may be present in substituents such as alkyl. All such isomers, as well as mixtures thereof, are included within the scope of the present disclosure.

When a fragment is absent, for example, when X in R—X is absent, it means that X is R.

Unless otherwise specified, the term “C₁₋₂₀ alkyl” by itself or in combination with other terms means, respectively, a straight or branched saturated carbon radical containing 1 to 20 carbon atoms. The C₁₋₂₀ alkyl group includes C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, and C₂₀ alkyl groups. The alkyl group can be monovalent (such as methyl), divalent (such as methylene), or polyvalent (such as methine). The C₁₋₂₀ alkyl includes, but is not limited to, methyl (Me), ethyl (Et), propyl (including n-propyl and isopropyl), butyl (including n-butyl, isobutyl, s-butyl, and t-butyl), pentyl (including n-pentyl, isopentyl, and neopentyl), hexyl, heptyl, octyl, nonyl, and decyl. The term “C₁₋₄ alkyl” includes, but is not limited to, methyl (Me), ethyl (Et), propyl (including n-propyl and isopropyl), and butyl (including n-butyl, isobutyl, s-butyl, and t-butyl). The term “C₁₋₃ alkyl” includes, but is not limited to, methyl (Me), ethyl (Et), and propyl (including n-propyl and isopropyl). Unless otherwise specified, the term “C₁₋₃ alkyl” refers to a straight or branched group containing I to carbon atoms. The C₁₋₃ alkyl includes C₁₋₃, C₁₋₂, C₁, C₂, and C₃ alkyl groups. The alkyl group can be monovalent (such as methyl), divalent (such as methylene), or polyvalent (such as methine). The C₁₋₃ alkyl includes, but is not limited to, methyl (Me), ethyl (Et), and propyl (including n-propyl and isopropyl). Unless otherwise specified, the “alkyl” is optionally substituted with I to 5 F, Cl, Br, I, OH, NH₂, or CN.

Unless otherwise specified, the term “aryl” refers to a polyunsaturated carbocyclic ring system, which may be a monocyclic, bicyclic, or polycyclic ring system, where at least one ring is aromatic. The individual rings in the bicyclic and polycyclic ring systems described are fused, which may be mono- or poly substituted and may be monovalent, divalent, or polyvalent. C₆₋₁₂ aryl includes, but is not limited to, phenyl and naphthyl (including 1-naphthyl and 2-naphthyl). Unless otherwise specified, the “aryl” is optionally substituted with 1 to 5 F, Cl, Br, 1, 01-1, NI1), or CN. “aryl-0-” refers to aryl bonded to the rest of a molecule via an oxygen bond (—O—). “aryl—NTI-” refers to aryl bonded to the rest of a molecule via a nitrogen bond.

Unless otherwise specified, the term “5- to 10-membered heteroaryl” refers to a 5- to 12-membered ring system group, including a hydrogen atom; 5 to 9 cyclic carbon atoms, 1 to 9 heterocyclic atoms selected from the group consisting of nitrogen, oxygen, and sulfur, and at least one aromatic ring containing a heteroatom. For a purpose of describing the examples in the present disclosure, heteroaryl may be a monocyclic, bicyclic, tricyclic, or tetracyclic ring system and may include fused or bridged ring systems. Nitrogen, carbon, or sulfur atoms in the heteroaryl group can be optionally oxidized; the nitrogen atoms can optionally be quaternized. Heteroaryl includes but is not limited to, azacycloheptatrienyl, acridinyl, benzimidazolyl, benzothiazolyl, benzindolyl, benzodioxolyl, benzofuranoyl, benzoxazolyl, benzothiazolyl, benzothiadiazolyl, benzo[b][1,4]dioxocyclohepta-5-enyl, 1,4-benzodioxanyl, benzonaphthofuryl, benzoxazolyl, benzo-m-dioxocyclopentenyl, 1,4-benzodioxyheterocyclohexaenyl, benzopyranyl, benzopyranonyl, benzofury , benzofbranonyl, benzothienyl, benzotriazolyl, benzo[4,6]imidazo[1,2-a]pyridyl, carbazolyl, cinnolinyl, dibenzofuryl, dibenzothienyl, furyl, furanonyl, isothiazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxypyridyl, 1-oxypyrimidyl, 1-oxypyrazinyl, 1-oxypyridazinyl, 1-phenyl-11-1-pyrrolyl, phenazinyl, phenothiazinyl, phenoxazinyl, phthalazinyl, pteridyl, purinyl, pyrrolyl, pyrazolyl, pyridyl, pyrazinyl, pyrimidinyl, pyridazinyl, quina.zolinyl, quinoxalinyl, quinolinyl, quinuclidinyl, isoquinolinyl, tetrahydroquinolyl, thiazolyl, thiadiazolyl, triazolyl, tetrazolyl, triazinyl, and thienyl.

The term “substituted” means that any one or more hydrogen atoms on a specified atom are substituted by a substituent, which may include deuterium and hydrogen variants, as long as the valence of the specified atom is normal and a substituted compound is stable. When the substituent is oxygen (═O), two hydrogen atoms are substituted. Oxygen substitution does not occur in aromatic groups.

The term “optionally substituted” means that it may or may not be substituted; unless otherwise specified, the kind and number of substituents can be arbitrary based on the desired chemical structure.

When any variable (such as R) occurs more than once in the composition or structure of a compound, its definition in each case is independent. Thus, for example, if a group is substituted with 0 to 2 R, the group may be optionally substituted with up to two R with independent options for R in each case. Furthermore, combinations of substituents and/or variants thereof are permissible only if such combinations result in stable compounds.

When a linking group is 0, such as —(CRR)₀—, it means that the linking group is a single bond.

In the present disclosure, the term “therapeutically effective amount” means: (i) an amount of a compound of the present application for treatment or prevention of a specific disease, condition, or disorder; (ii) an amount of a compound of the present application for alleviating, ameliorating, or eliminating one or more symptoms of the specific disease, condition, or disorder; or (iii) an amount of a compound of the present application that prevents or delays the onset of the one or more symptoms of the specific disease, condition, or disorder described herein. The “therapeutically effective amount” of the compound of the present application varies depending on the compound, the disease state and its severity, the mode of administration, and the age of the mammal to be treated, which can also be routinely determined by those skilled in the art based on their knowledge and the present disclosure.

In the present disclosure, the term “pharmaceutical composition” refers to a mixture composed of one or more compounds of the present application or salts thereof and a pharmaceutically acceptable carrier. The pharmaceutical composition facilitates the administration of a compound of the present application to an organism.

In the present disclosure, the term “pharmaceutically acceptable carrier” refers to those adjuvants that have no obvious irritating effect on the organism and do not impair the biological activity and performance of the active compound. Such suitable adjuvants are well known to those skilled in the art, including carbohydrates, wax, water-soluble and/or water-swellable polymers, hydrophilic or hydrophobic materials, gelatin, oils, solvents, water, liposomes, polymeric micelles, or inorganic nanocarriers.

In the present disclosure, the solvent is commercially available.

In the present disclosure, the following abbreviations are used: HIF-1α stands for hypoxia-inducible factor-1α; PD-L1 stands for programmed death receptor-ligand 1; CD47 stands for cluster of differentiation 47; MO stands for indoleamine 2,3-dioxygenase; P-gp stands for P-glycoprotein; DOX stands for doxorubicin; DMSO stands for dimethyl sulfoxide; PBS stands for phosphate-buffered saline; EDTA stands for ethylenediaminetetraacetic acid.

In the present disclosure, the compounds are named according to a conventional nomenclature in the art or by ChemDraw® software, and the commercially available compounds are named according to the supplier's catalog.

### BRIEF DESCRIPTION OF THE DRAWINGS

FIG. 1 shows a Western blot result that the compound of the present disclosure reduces PD-Li expression of CT26 cells in Test Example 1A;

FIG. 2 shows a Western blot quantitative result that the compound of the present disclosure reduces PD-L1 expression of CT26 cells in Test Example 1A;

FIG. 3 shows a PCR result of the compound of the present disclosure in reducing mRNA expression of PD-L1 in C₁₂₆ cells in Test Example 1B;

FIG. 4 shows a flow cytometry result of reduction on PD-L1 expression in C₁₂₆ cells by different concentrations of compound 7 (ROX) in Test Example 1C;

FIG. 5 shows a result of increasing immunogenic death of C₁₂₆ cells after compound 7 (ROX) in Test Example 1D is used in combination with DOX, which is compared with the DOX alone;

FIG. 6 shows an expression of related genes in mouse macrophages treated with the compound 7 (ROX) in Test Example 1E, which is compared with untreated mouse macrophages;

FIG. 7l shows an experimental result that the compound of the present disclosure in Test Example 1F inhibits the production of kynurenine in CT26 cells;

FIG. 8 shows the cytotoxicity of the compound of the present disclosure in Test Example 2A used in combination with a chemotherapeutic drug DOX on CT26 cells;

FIG. 9 shows an experimental result of cytotoxicity of the compound of the present disclosure in Test Example 2A;

FIG. 10 shows an experimental result of cytotoxicity of compound 7 (ROX) in Test Example 2A in combination with the chemotherapeutic drug paclitaxel (PTX);

FIG. 11 shows an experimental result of cytotoxicity of compound 7 (ROX) in Test Example 2A in combination with the chemotherapeutic drug oxaliplatin (OXA);

FIG. 12 shows an experimental result of cytotoxicity of compound 7 (ROX) in Test Example 2A in combination with the chemotherapeutic drug camptothecin (irinotecan, SN38);

FIG. 13 shows a result of the compound of the present disclosure in Test Example 2B in reducing an expression of HIF-1α in CT26 cells;

FIG. 14 shows a result of endocytosis def rhodatnine 123 (Rh123) in CT26 cells treated with the compound 7 (ROX) in Test Example 2C, which is compared with untreated cells;

FIG. 15 shows a tumor inhibition curve of the compound 7 (ROX) in Test Example 3, the DOX, and a combination thereof on a tumor model of CT26 tumor-bearing mice;

FIG. 16 shows a body weight curve of mice treated by the compound 7 (ROX) in Test Example 3, the DOX, and a combination thereof;

FIG. 17 shows a tumor inhibition curve of the tumor model of the CT26 tumor-bearing mice after the combination of a doxorubicin liposome (Doxil) and a compound 7 liposome (Roxil) in Test Example 4;

FIG. 18 shows a body weight curve of mice treated by the doxorubicin liposome (Doxil) and compound 7 liposome (Roxil) in Test Example 4;

FIG. 19 shows a tumor inhibition curve of the tumor model of the CT26 tumor-bearing mice after the combination of a doxorubicin liposome (Doxil) and a compound 7-loaded micelle (PEG-PLA-ROX) in Test Example 5; and

FIG. 20 shows a body weight curve of mice treated by the doxorubicin liposome (Doxil) and the compound 7-loaded micelle (PEG-PLA-ROX) in Test Example 5.

### DETAILED DESCRIPTION OF THE EMBODIMENTS

The present disclosure will be further described below in conjunction with the example. It should be noted that the following descriptions are only intended to explain the present disclosure and do not limit the content thereof. In the present disclosure, the compounds can be prepared by a variety of synthetic methods well known to those skilled in the art, including the specific examples listed below, the examples by combining the compounds with other compound synthesis methods, and equivalent alternatives well-known to those skilled in the art, and are also commercially available. Preferred examples include, but are not limited to, the examples of the present disclosure. It will be apparent to those skilled in the art that various changes and modifications can be made to the specific examples of the present disclosure without departing from the spirit and scope of the present disclosure.

### Example 1

### Preparation Route of the Esterified Derivative of N-(3-hydroxypyridine-2-carbonyl)glycine

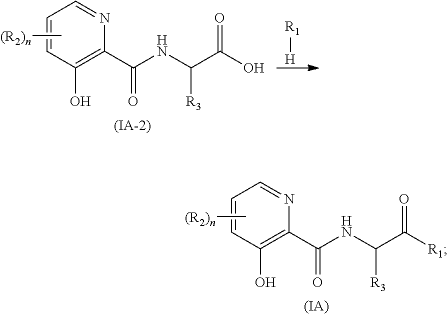

R₁ is —O—C₁₋₂₀ alkyl or —O—C₆₋₁₂ aryl.

A compound (2) was taken as an example.

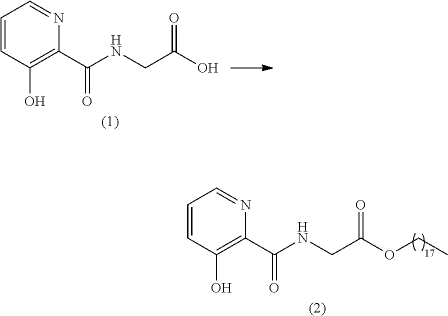

A compound (1) (200 mg, 1.02 mmol) was dissolved in 20 mL of dry N, N-dimethylformamide, and oleyl alcohol (329 mg, 1.22 mmol) and 4-dimethylaminopyridine (6.23 mg, 0.05 mmol) was added. Dicyclohexylcarbodiimide (252 mg, 1.22 mmol) was added dropwise under N2 protection in an ice bath and stirred for 1 h; the ice bath was removed, and the reaction was continued overnight. After the reaction, the solvent was removed by rotary evaporation under reduced pressure, and purification was conducted by a silica gel column and a mobile phase of n-hexane: ethyl acetate=5:1 to obtain the compound (2) as a white solid with a yield of 86.9%. Formula: [C₂₆H₄₅N₃O₃]⁺, Calc. 448.33, found 448.23.

### Example 2

### A Preparation Route of an Amidated Derivative of N-(3-hydroxypyridine-2-carbonyl)glycine

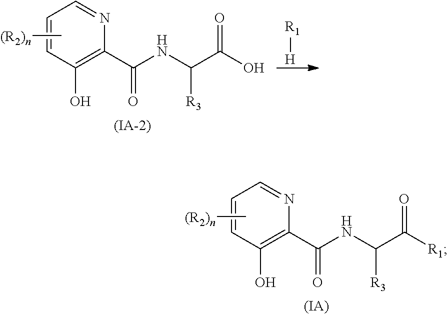

R₁ is NH₂ or —NH—C₁₋₂₀.

A compound (3) was taken as an example.

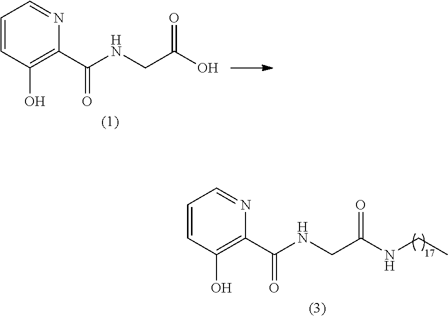

The compound (1) (200 mg, 1.02 mmol) was dissolved in 20 ml, of dry N, N dimethylformamide, and 1-(3-dimethylaminopropyl)-3-ethylcarbodiimide HCl (603 mg, 3.06 mmol), 1-hydroxybenzotriazole (207 mg, 1.53 mmol.), and oleylamin.e (328 mg, 1.23 mmol) were added. N,N-diisopropylethylamine (527 mg, 4.08 mmol) was added dropwise under N₂, protection in an ice bath and stirred for 1 h; then the ice bath was removed, and the reaction was continued overnight. After the reaction, most of the solvent was removed by rotary evaporation under reduced pressure, redissolved in ethyl acetate, and then washed with 1N hydrochloric acid and saturated brine; after drying and concentration, purification was conducted by a mobile phase of n-hexane: ethyl acetate=3:1 through a column, and a purified product was dried to obtain the pale yellow solid compound (3) with a yield of 91.2%. Formula: [C₂₆H₄₅N₃O₃]⁺, Calc. 447.35, found 447.15.

in the present disclosure, the compound could be prepared by referring to the literature (Dynamic combinatorial mass spectrometry leads to inhibitors of a 2-oxoglutarate-dependent nucleic acid demethylase. Journal of Medicinal Chemistry, 2012, 55 (5): 2173) or was obtained commercially (the small compound used in the present disclosure was from MedChemExpress China).

### Test Example 1

### The Compound of the Present Disclosure as an Immune Sensitizer

Configuration method: DMSO solutions of the compounds of the present disclosure obtained in Examples 1 and 2 could be directly used in cell experiments.

### Test Example 1A

### The Compounds of the Present Disclosure in Reducing Expression of PD-L1 in Tumor Cells Detected by Western Blot

CT26 cells were inoculated in a 6-well plate at 2×104 cells per well and placed in a 37° C. incubator. After the cells adhered, DMSO solutions (5 μM) of compounds 1 to 8 were added separately, and the incubation was continued for 48 h. The medium was discarded, the cells were rinsed three times with pre-cooled PBS, and the washing solution was discarded. 0.2 nrii, of a cell lysis solution containing a protease inhibitor was added to each well and placed on ice to conduct lysis for 30 min.

After the lysis was completed, a lysate and cell debris were scraped to one side of the Petri dish with a cell scraper, the lysate was transferred to a 1.5 mL Ep tube with a pipette, centrifuged at 4° C. (12,000 rpm/5 min), and a supernatant was collected to determine its protein concentration.

The protein sample was diluted with a 1UPAlysis buffer to a certain concentration, 5×SDS loading buffer was added in a protein amount of 1 μg/μL, incubated on a metal bath at 95° C. for 5 min, cooled to room temperature, and the sample was loaded to an SDS-PAGE electrophoresis gel (5% for stacking gel and 12% for resolving gel). Under a voltage of 90 V, the protein was run to the bottom of the stacking gel to form a straight line, then the voltage was adjusted to 125 V, and the electrophoresis was stopped after the bromophenol blue appeared.

The protein was then transferred to a nitrocellulose membrane at a voltage of 80 V for 90 min. The membrane was blocked in 5% skim milk for one hour at room temperature. The membrane was washed three times with a TBST buffer, and corresponding primary antibodies (anti-PD-L1, 1:2000; anti-GAPDH, 1:10000) were added to the membrane separately and incubated at 4° C. overnight. The membrane was washed three times with the TBST buffer, and a horseradish peroxidase-labeled secondary antibody was added to the membrane and incubated at room temperature for 1 h. The membrane was washed thoroughly, subjected to chemiluminescence color development, and photographed with a chemiluminescence imager.

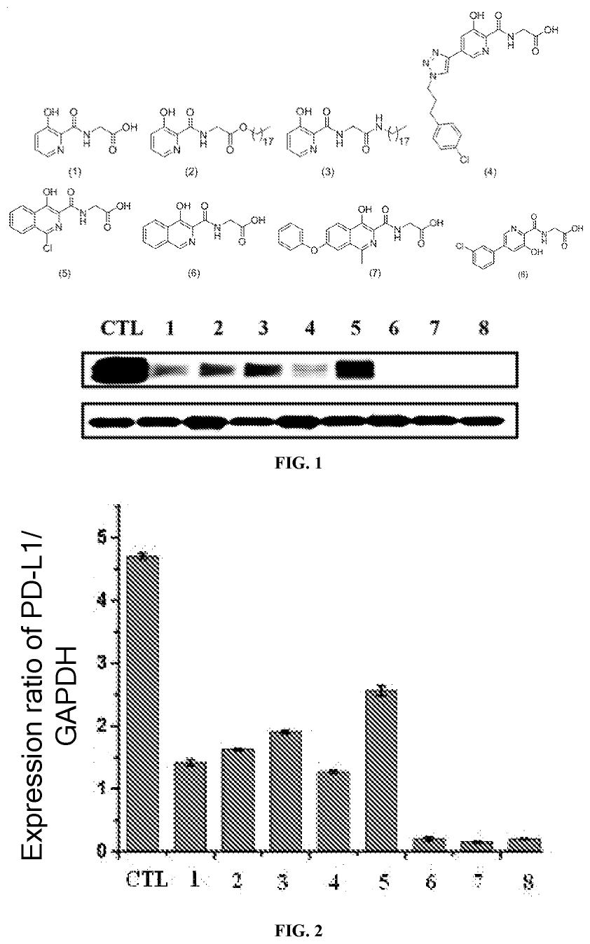

The results were shown in FIG. 1 to FIG. 2, the compounds 1 to 8 of the present disclosure could reduce the expression of PD-L1 in tumor cells, where compounds 6, 7, and 8 had the best effect. It was proved that the compound of formula (I) in the present disclosure could reduce the expression of PD-L1 in tumor cells.

### Test Example 1B

### The Compounds of the Present Disclosure in Reducing mRNA Expression of PD-L1 in Tumor Cells Detected by gPCR

CT26 cells were inoculated in a 6-well plate at 2×10⁵ cells per well and placed in a 37° C. incubator. After the cells adhered, DMSO solutions (5 μM) of the compounds of the present disclosure were added separately, and the incubation was continued for 48 h. Total RNA was lysed and extracted, and reverse transcription and PCR experiments were conducted to detect an RNA level of PD-L1 in CT26 cells, using a GAPDH gene as an internal reference.

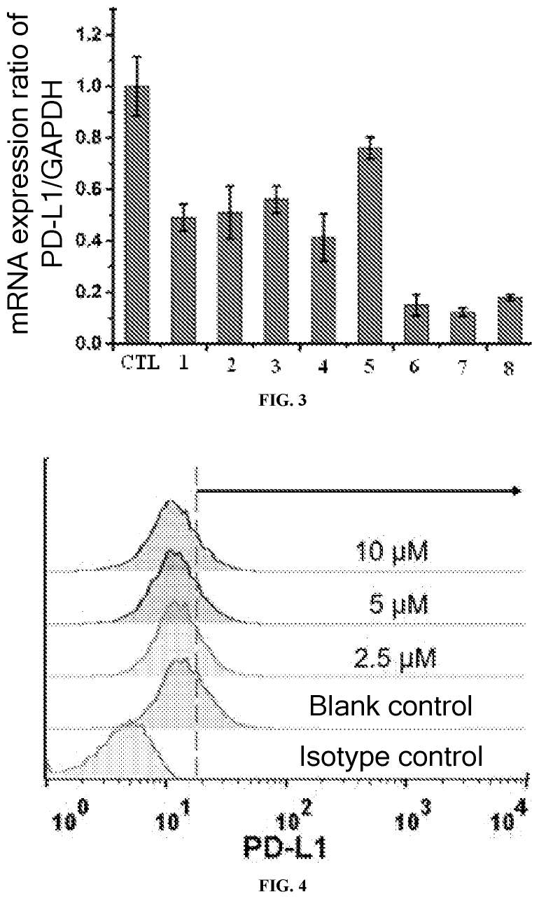

The test results are shown in FIG. 3. The mRNA expression level of PD-L1 was significantly reduced after adding the compound of the present disclosure, indicating that the compound of the present disclosure could reduce the mRNA expression of PD-L1 in tumor cells. Among them, compounds 6, 7, and 8 had the best effect.

### Test Example 1C

### The Compounds of the Present Disclosure in Reducing Expression of PD-L1 in Tumor Cells Detected by Flow Cytometry

CT26 cells were inoculated in a 6-well plate at 2×10⁴ cells per well. After the cells adhered. DMSO solutions (2.5 μM, 5 μM, and 10 μM) of the compounds of the present disclosure were added separately, and the incubation was continued for 24 h. The medium was discarded, the cells were rinsed three times with PBS, and 0.2 mL of trypsin containing EDTA was added to each well. The digested cells were collected in a flow tube, and a supernatant was removed by centrifugation. The cells were resuspended in a PBS containing 5% goat serum, and an anti-mouse PD-L1 primary antibody (1 μg/1×10⁶ cells) was added, and the cells were incubated at 4° C. for 30 min and washed three times with PBS. An equivalent amount of APC-labeled goat anti-rabbit secondary antibody was added and incubation was continued for 30 min; the cells were washed 3 times with PBS and subjected to flow detection on-machine. The results were shown by taking compound 7 as an example.

The results are shown in FIG. 4. The integral value in the gray area represented a PD-L1 expression rate, and the blank group had a PD-L1 expression rate of 32.7%. The 2.5 compound 7 group had a PD-L1 expression rate of 24%, the 5 1μM compound 7 group had a PD-L1 expression rate of 20.8%, and the 10 μM compound 7 group had a PD-L1 expression rate of 18.8%. It showed that compound 7 could significantly reduce the expression of PD-L1 in tumor cells in a concentration-dependent manner.

### Test Example 1D

### The Compounds of the Present Disclosure in Enhancing Drug-Induced Immunogenic Death of Tumor Cells

CT26 cells were plated in confocal culture dishes, and after overnight cell adherence, different drug treatment groups were added, and the incubation was continued for 24 h. The medium was discarded, the cells were rinsed 3 times with PBS, and fixated with 4% paraformaldehyde for 10 min. The cells were rinsed 3 times with PBS for 3 min each time, a 3% BSA solution was added for blocking for 30 min at 37° C., and the blocking solution was removed with absorbent paper. 200 μuL of a calreticulin antibody (FITC-anti-CRT, 1:200) was added to each well and incubated at room temperature for I h in the dark. The cells were rinsed with PBS 3 times for 3 min each time, then a DAPI staining solution was added to each dish and incubated in the dark for 5 min. After washing three times with PBS, the cells were observed under a laser con focal microscope.

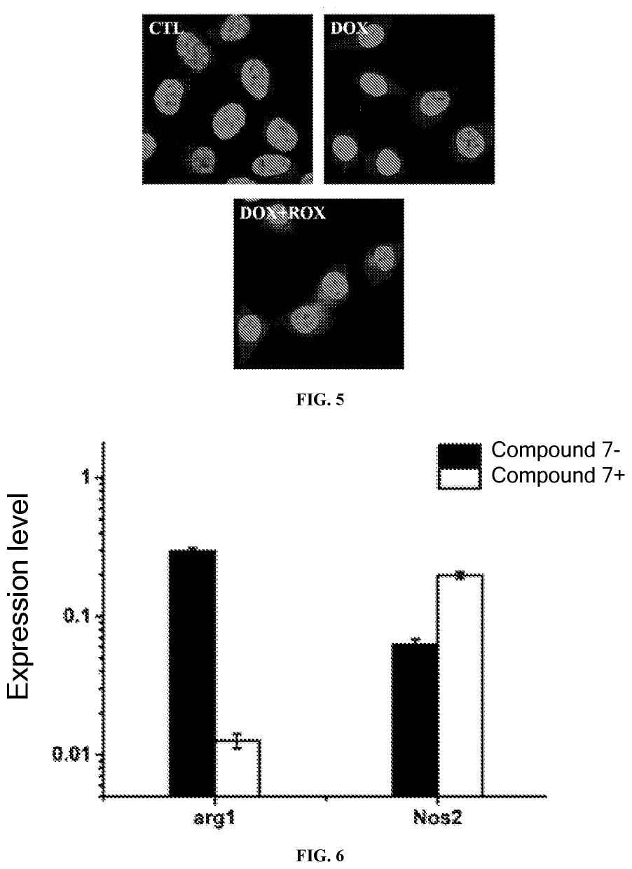

The results are shown with doxorubicin (DOX) in combination with compound 7 (FIG. 5). The compound 7 could make doxorubicin-induced CT26 tumor cells express more calreticulin, indicating more immunogenic death.

### Test Example 1E

### The Compounds of the Present Disclosure in Promoting Polarization of Macrophages from M2 to M1

A mouse macrophage cell line Raw264.7 was inoculated in a 24-well plate. After 12 hours of cell adhesion, the cells were further cultured for one day in a medium containing IL-4 (40 ng/mL) to induce the cells to differentiate into M2 tumor-associated macrophages (TAM2s). DMSO solutions (5 μM) of different groups of the compounds of the present disclosure were added to the TAM2s to treat for 24 h, the cells were collected and lysed, and a total RNA was extracted. The total RNA was subjected to reverse transcription and PCR experiments to detect the RNA levels of an M2 macrophage-specific protein argl and an M1 macrophage-specific protein Nos2 with an hprt gene as an internal reference.

Compound 7 was taken as an example to show the results in FIG. 6. Compared with TAM2s not treated with compound 7 of the present disclosure, argl was significantly decreased and Nos2 was significantly increased after adding compound 7, indicating that the compound of the present disclosure could promote the polarization of macrophages from M2 to MI. The M2 macrophages that promote tumor growth were transformed into tumor-suppressing M1 macrophages, which was beneficial to improve a tumor treatment effect.

### Test Example 1F

### The Compounds of the Present Disclosure in Inhibiting Expression of Indoleamine 2,3-dioxygenase

CT₂₆ cells were inoculated in a 12-well plate at 5×10⁴ cells/well, and 2 ml, of a medium (containing 100 μM tryptophan) was added to each well. After culturing for one day, a certain concentration gradient of the DMSO solution of the compound of the present disclosure was added, and then 0.1 μg/mL of INF-7 was added to induce the expression of IDO. After 72 h of incubation, 200 μL of a supernatant was added to 10 μL of a 30% trifluoroacetic acid (TFA) solution to precipitate proteins. A content of kynurenine in the supernatant solution was detected by HPLC, and each well was repeated three times.

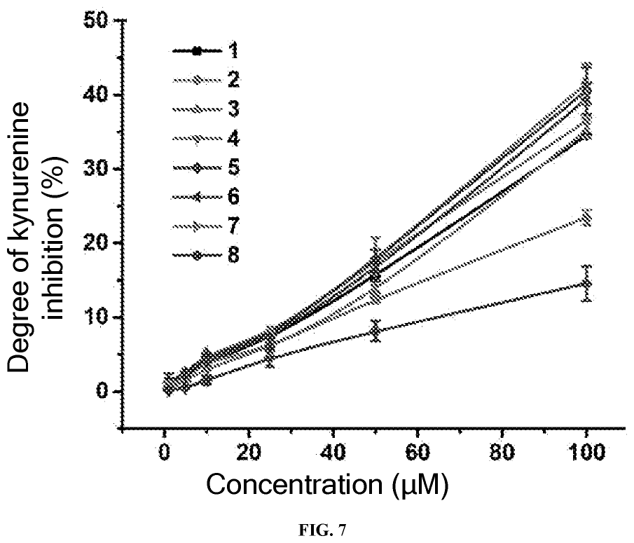

The results as shown in FIG. 7 proved that the compounds of the present disclosure could inhibit the conversion of tryptophan to kynurenine and indicated that the compounds of the present disclosure could inhibit the expression of indoleamine 2,3-dioxygenase. Therefore, the compounds of the present disclosure could enhance the immunotherapy effect of cancer by inhibiting the expression of indoleamine 2,3-dioxygenase.

### Test Example 2

### Use of the Compound of the Present Disclosure as a Chemosensitizer

Configuration method: DMSO solutions of the compounds of the present disclosure obtained in Examples 1 and 2 could be directly used in cell experiments.

### Test Example 2A

### Cytotoxicity Study of the Compounds of the Present Disclosure in Combination with Various Chemotherapeutic Drugs

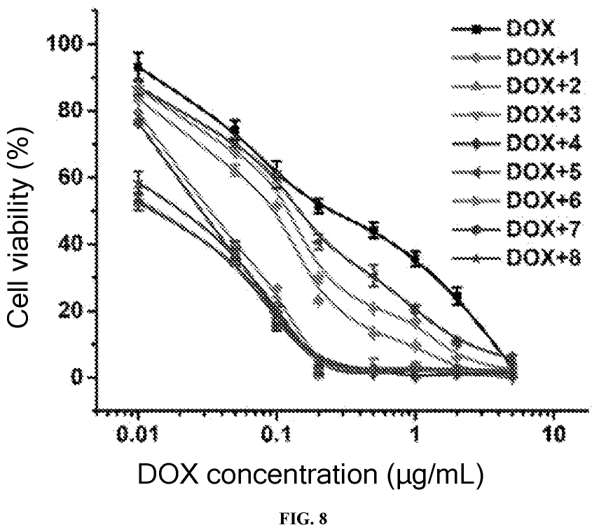

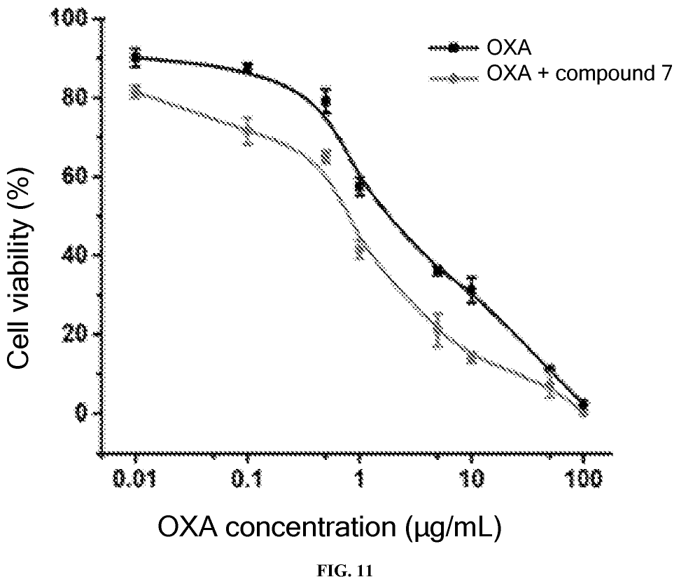

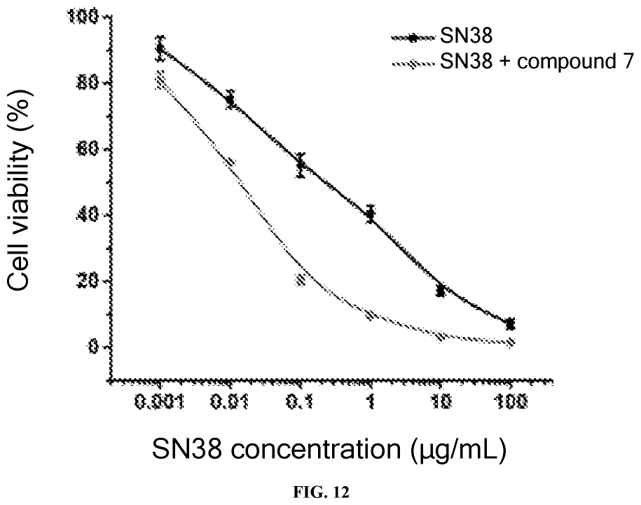

CT26 cells (MC₃₈ cells, 4T1 cells, B16FIO cells, HePa1-6 cells, H22 cells, LLC cells, MB49 cells, P388 cells, C₆ cells, BXPC-3 cells, Hela cells, MDA-MB-231 cells, A2780 cells. PC₃ cells. HepG2 cells, and HGC-27 cells) were cultured at 5,000 cells/well in a 96-well plate, 100 μL of a medium was added to each well, and the cells were incubated in a 37° C. constant-temperature incubator with 5% CO₂ and 95% humidity for 24 h. 100 μL of different concentrations of drugs (DOX: 0.01 μg/mL to 10 μg/mL; PTX: 0.01 μg/mL to 50 μg/mL; Cela: 0.05 μg/mL to 1 μg/mL; sensitizer: 5 μg/mL) were added to each well, while 100 lit of a medium solution was added to the blank group. After culturing for 48 h, centrifugation was conducted at 1,100 rpm for 6 min, the medium in each well was discarded, 100 μL of an 11,11T working solution was added, and the culturing was continued for 3 h. Centrifugation was conducted at 3,300 rpm. for 5 min, the MITT medium in each well was discarded, 100 pt, of the DMSO was added, and the plate was shaken for 5 min to dissolve all crystals in each well. An absorbance of the sample at 562 nm was detected by a microplate reader. Each set of data was an average of three independent experimental results on the same sample. The results are shown in FIG. 8 to FIG. 12.

FIG. 8 showed that a combination of the compounds of the present disclosure and chemotherapeutic drugs could greatly reduce cell viability compared with the chemotherapeutic drugs alone, and compound 7 had the best effect.

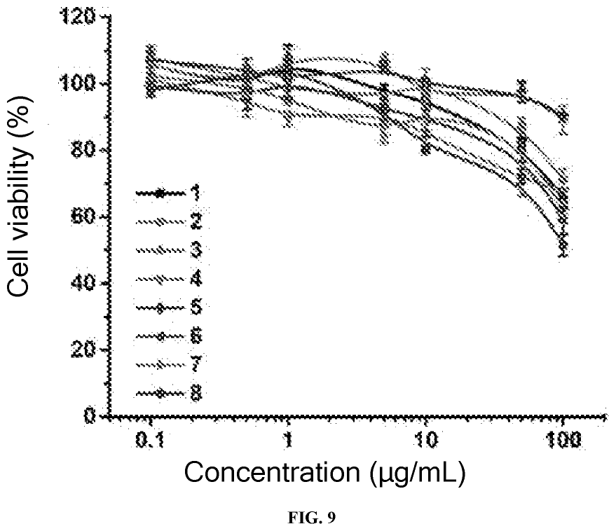

FIG. 9 showed that at 0.1 μ/mL, to 10 μg/mL, the compounds of the present disclosure did not have significant cytotoxicity.

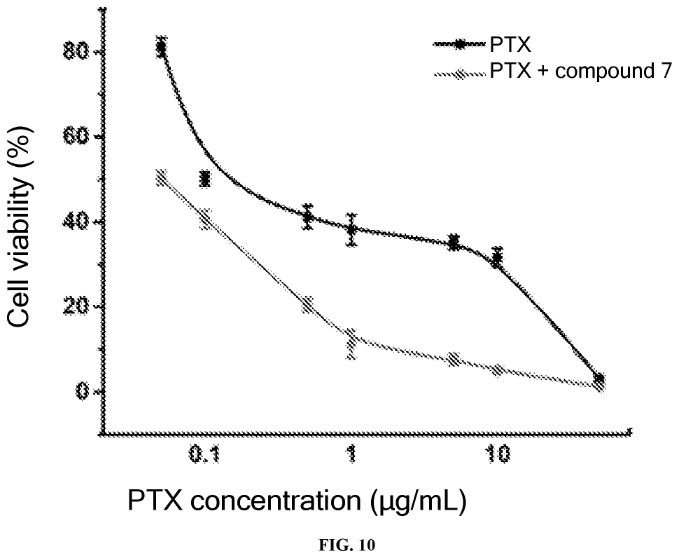

FIG. 10 to FIG. 12 showed that compound 7 of the present disclosure could increase the toxicity of doxorubicin (DOX), paclitaxel (PTX), oxaliplatin (OXA), or camptothecin (irinotecan, SN38) on CT26 cells.

Table 1 showed experimental results of the cytotoxicity of compound 7 in combination with the chemotherapeutic drug DOX on various cell lines. It was shown that the combination of compound 7 and DOX could greatly reduce the survival rate of various tumor cells.

TABLE 1

| | IC₅₀ (μg/mL) | |
|---|---|---|
| Cell name | DOX | DOX + ROX |
| CT26 mouse colon cancer cells | 0.256 ± 0.017 | 0.014 ± 0.001 |
| MC38 mouse colon cancer cells | 0.411 ± 0.017 | 0.019 ± 0.001 |
| 4T1 mouse breast cancer cells | 1.756 ± 0.221 | 0.314 ± 0.014 |
| B16F10 mouse melanoma cells | 0.214 ± 0.011 | 0.043 ± 0.005 |
| HePa1-6 mouse hepatoma cells | 1.349 ± 0.331 | 0.521 ± 0.077 |
| H22 mouse hepatoma cells | 5.127 ± 0.119 | 1.421 ± 0.781 |
| LLC mouse lung cancer cells | 0.088 ± 0.007 | 0.058 ± 0.001 |
| MB49 mouse bladder cancer cells | 0.447 ± 0.022 | 0.171 ± 0.011 |
| P388 mouse lymphoma cells | 0.197 ± 0.029 | 0.088 ± 0.014 |
| C6 rat glioma cells | 0.119 ± 0.026 | 0.072 ± 0.017 |
| BXPC-3 human pancreatic cancer | 14.227 ± 1.677 | 8.375 ± 0.537 |
| cells | | |
| Hela human cervical cancer cells | 2.771 ± 0.492 | 1.872 ± 0.513 |
| MDA-MB-231 human breast cancer | 3.572 ± 0.153 | 0.379 ± 0.017 |
| cells. | | |
| A2780 Human ovarian cancer cells | 10.111 ± 1.241 | 3.124 ± 0.861 |
| PC3 human prostate cancer cells | 2.322 ± 0.195 | 1.177 ± 0.119 |
| HepG2 human hepatoma cells | 3.454 ± 0.778 | 2.551 ± 0.541 |
| HGC-27 human gastric cancer cells | 1.819 ± 0.227 | 0.105 ± 0.007 |

Table 2 showed experimental results of the cytotoxicity of compound 7 in combination with various chemotherapeutic drugs on the CT26 cell line. It was shown that compound 7 could increase the toxicity of various chemotherapeutic drugs to 0126 cells.

TABLE 2

IC₅₀ (μg/mL)

| Drug name | Compound 7− | Compound 7+ |
|---|---|---|
| Paclitaxel | 0.297 ± 0.022 | 0.077 ± 0.007 |
| Cis-platinum | 1.281 ± 0.146 | 0.571 ± 0.068 |
| Carboplatin | 0.761 ± 0.044 | 0.192 ± 0.052 |
| Oxaliplatin | 0.650 ± 0.009 | 0.197 ± 0.022 |
| Eloxatin | 4 774 ± 0.891 | 2.497 ± 0.185 |
| Mitoxantrone | 2.117 ± 0.297 | 0.815 ± 0.021 |
| Gambogic acid | 2.179 ± 0.454 | 0.792 ± 0.181 |
| Tripterine | 5.219 ± 0.242 | 1.592 ± 0.142 |
| Irinotecan | 2.415 ± 0.197 | 1.415 ± 0.022 |
| 7-ethyl-10- | 0.066 ± 0.006 | 0.025 ± 0.001 |
| hydroxycamptothecine | | |
| Docetaxel | 0.377 ± 0.015 | 0.029 ± 0.005 |
| Vincristine | 68.179 ± 2.291 | 44.312 ± 1.851 |
| Epirubicin | 97.442 ± 3.331 | 17.125 ± 1.338 |

### Test Example 2B

### The Compounds of the Present Disclosure in Reducing Expression of HIF-1α in Tumor Cells Detected by Flow Cytometry

CT26 cells were evenly plated in a 6-well plate at 2×10⁴ cells per well. After the cells adhered, DMSO solutions (5 μM) of the different compounds of the present disclosure were added separately, and the incubation was continued for 24 h. The medium was discarded, the cells were rinsed three times with PBS, and 0.2 mL of trypsin containing EDTA was added to each well. The digested cells were collected in a flow tube, and a supernatant was removed by centrifugation; the cells were resuspended in a PBS containing 5% goat serum, and an FITC anti-mouse HIF-1α antibody (1 μg/1×10⁶ cells) was added, incubated at 4° C. for 30 min, and washed three times with PBS, and subjected to flow detection on-machine.

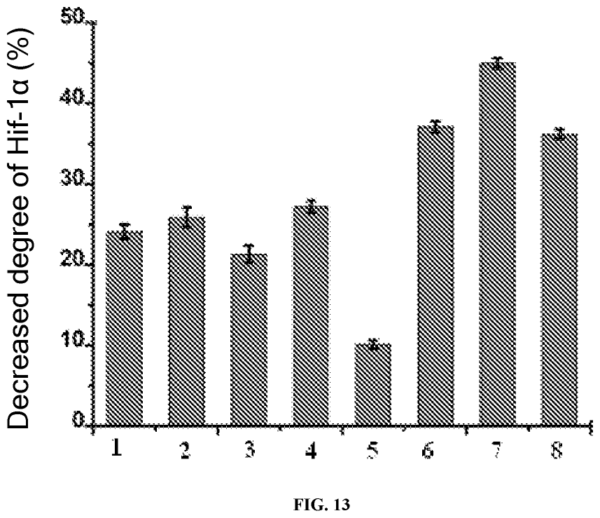

The results are shown in FIG. 13, compared with the blank control, N-(3-hydroxypyridine-2-carbonyl)glycine and derivatives thereof could significantly reduce the expression of HIF-1α in tumor cells, where compounds 6, 7, and 8 had the best effect and the compound 7 reduced HIF-1α by 45% at 5 μM.

### Test Example 2C

### The Compounds of the Present Disclosure in Enhancing the Ability of Rhodatnine 123 (Rh123) to Enter Tumor Cells

Rh123 is a substrate of a multidrug resistance protein P-gp. The decreased expression of P-gp can reduce the cellular efflux of Rh123 and increase its intracellular content, such that this substrate can measure the activity of P-gp protein in cells. CT26 cells were inoculated in a confocal imaging dish at 1×10⁴ cells/well and incubated in a 37° C. constant-temperature incubator for 24 h. Each well was replaced with a fresh medium, and an Rh123 solution (1 uM) and the DMSO solution (1 μM) of the compound 7 of the present disclosure were added, the cells were incubated for 6 h, and intracellular conditions of the Rh123 were observed by confocal microscope. Rh123 has an excitation wavelength of 488 nm and an emission wavelength of 500 nm to 550 nm.

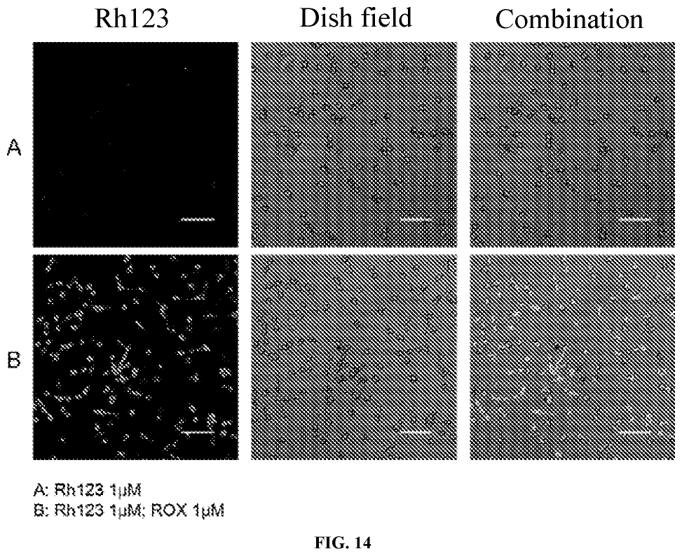

The results are shown in FIG. 14. Adding compound 7 could significantly increase an intracellular concentration of the Rh123, proving that the efflux of Rh123 could be reduced by inhibiting P-gp.

### Test Example 3

### The Compound of the Present Disclosure in Enhancing Antitumor Activity of Drugs In Vivo

(1) Preparation Method

Take compound 7 as an example: 20 mg of compound 7 was dissolved in 0.5 ml, of DMSO, 0.5 mL of polyoxyethylene castor oil, Tween 80, or polyethylene glycol 500 was added (the polyoxyethylene castor oil was used in the present disclosure), and then mixedby wellthe vortex, The obtained mixed solution was added to 9 mL of the PBS, and an injection solution of compound 7 was obtained by mixing. The injection could be stored at 4° C. for more than 6 months without precipitation of a solid powder.

(2) Tumor Inhibition Experiment

The tumor inhibition of compound 7 in combination with chemotherapeutic drugs (doxorubicin, paclitaxel, gemcitabine, oxaliplatin, camptothecin derivatives, irinotecan, tripterine, and gambogic acid) was investigated on colon cancer in C₁₂₆ mice. Balb/c mice were subcutaneously inoculated with 1×10⁶ CT26 tumor cells, and after the tumor grew to about 80 mm³, tail vein injection was conducted every two days (on Day 0, Day 2, and Day 4). Taking the combination of compound 7 with doxorubicin hydrochloride as an example, there was a blank control group, a compound 7 group, a doxorubicin hydrochloride group, and a compound 7+ doxorubicin hydrochloride group (D1R_{1: 3} mg/kg DOX+5 mg/kg compound 7; D1R_{1 3} mg/kg DOX+10 mg/kg compound 7; D₁R_{3: 5} mg/kg DOX+5 mg/kg compound 7; D1R_{4: 5} mg/kg DOX+10 mg/kg compound 7;). After the administration, the mice were observed for another 12 d.

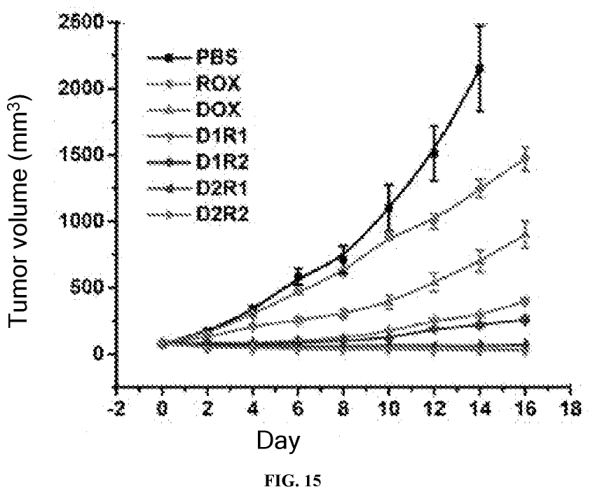

The results in FIG. 15 showed that, compared with compound 7 or doxorubicin hydrochloride alone, the tumor volume in the combination treatment group showed a decreasing trend, and the tumor did not grow after drug withdrawal and remained unchanged. It showed that the combination of compound 7 and doxorubicin hydrochloride exerted a more significant anticancer activity and had a certain memory after treatment. In addition, compound 7 could increase the tumor inhibition rate of other antitumor drugs by 30% to 70%, which had a therapeutic effect at the leading level in the field, showing a desirable prospect for use.

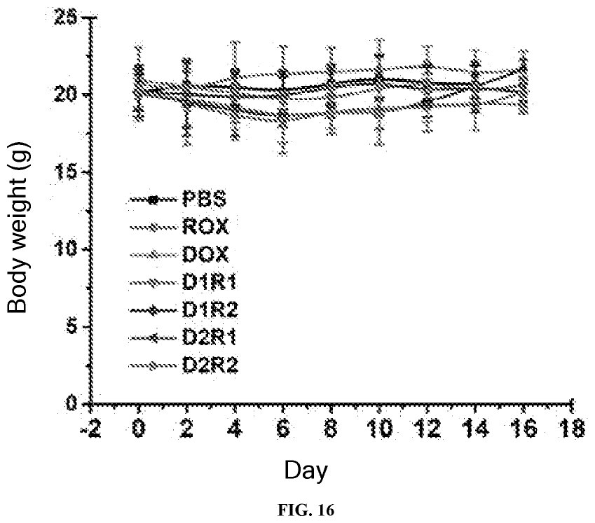

FIG. 16 showed that the body weight of mice in each group did not decrease, indicating that the drug had high biosafety and fewer side effects.

### Test Example 4

### Preparation of Liposome Composition of Compound 7 and Anti-Tumor Activity Experiments Thereof in Combination with Chemotherapeutic Drugs

A liposome preparation of compound 7 was prepared by a thin film dispersion method.

Step 1: 12.89 g of dioleoyl-phosphatidyl ethanolamine (DOPE), 2.11 g of cholesterol hemisuccinate (CHEMS), 6.52 g of distearoylphosphatidyl ethanolamine-polyethylene glycol 2000 (DSPE-mPEG2000), and 5 g of the compound 7 were dissolved in chloroform (12 mL) and methanol (4 mL) and spin-dried under reduced pressure in a water bath at 37° C. to form a membrane.

Step 2: 5 mL of deionized water or a buffer solution (a 1×PBS solution was used in the present disclosure) was added to the liposome membrane obtained in step 1 and hydrated at 4° C. to 60° C. for 24 h.

Step 3: The obtained solution was dialyzed in a dialysis bag for 8 h to obtain a liposome preparation (Roxil) of compound 7.

The liposome could be prepared from different kinds of lipid raw materials, and the desired solvent for the lipid raw material, such as DCM, chloroform, or methanol, could be selected as required. In this test example, chloroform: methanol=3:1 (12 mL and 3 mL) was selected. The concentration of N-(4-Hydroxy-1-methyl-7-phenoxyisoquinoline-3-carbonyl)glycine could be adjusted according to actual needs.

Size characterization of a pharmaceutical preparation: The particle size and distribution of the pharmaceutical preparation were determined by dynamic light scattering (DLS). The lipid preparation was assembled in water into nanoparticles with a dynamic size distribution of 0.110 and an average size of 105.9 nm. The size could be adjusted by liposome composition, preparation method, and the like.

Antitumor activity test:

Balb/c mice were subcutaneously injected with 1×10⁶ CT26 tumor cells, and after the tumor grew to about 80 mm³, tail vein injection was conducted every two days (on Day 0, Day 2, and Day 4) for administration. Taking a combination of Roxil and doxorubicin liposome as an example, there was a blank control group, a doxorubicin liposome group (Doxil, the DOX dosage was 5 mg/kg), a compound 7 doxorubicin liposome group (Roxil+Doxil, the DOX dosage was 5 mg/kg, the ROX dosage was 7.5 mg/kg), and a compound 7 liposome group (the ROX dosage was 7.5 mg/kg). After the administration, the mice were observed for another 18 d.

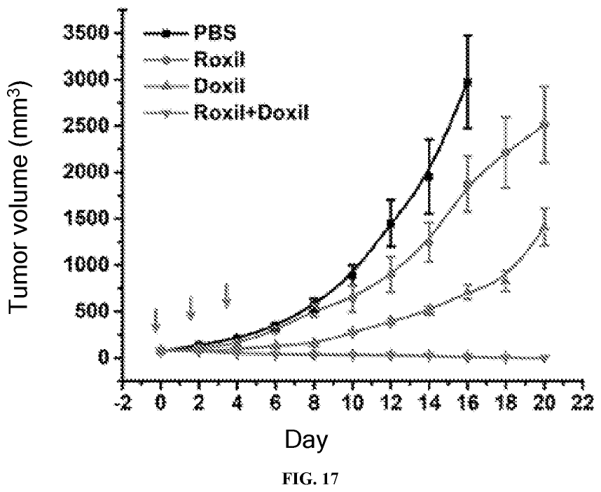

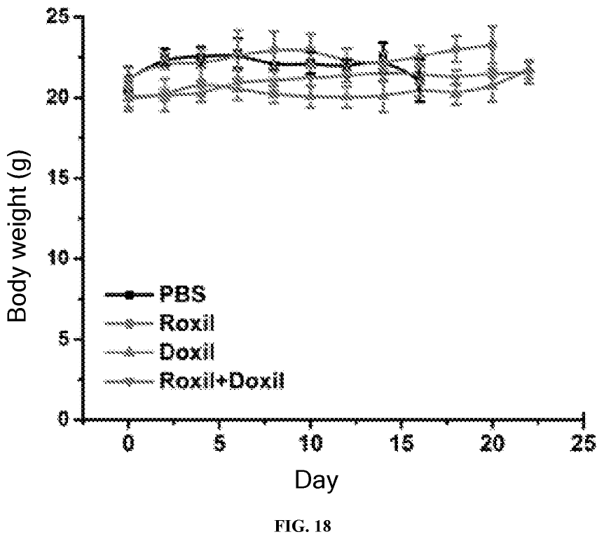

The results are shown in FIG. 17. The combination of the two preparations had an anti-tumor effect significantly better than that of the doxorubicin liposome alone. After 20 d, the tumors in the combination group were eliminated, while the tumors in the doxorubicin liposome group still showed a growth trend. FIG. 18 showed that the body weight of mice in each group did not decrease, indicating that the drug had high biosafety and fewer side effects.

After being prepared into a pharmaceutical preparation, the combination of compound 7 with chemotherapeutic drugs showed a significant anticancer activity, which had the greatest therapeutic effect, showing a desirable prospect for use.

### Test Example 5

### Preparation of Polymeric Micelle Composition of Compound 7 and Anti-Tumor Activity Tests Thereof in Combination with Chemotherapeutic Drugs

A polymeric micelle preparation of compound 7 was prepared by a thin film evaporation method.

Step 1: Compound 7 (5 mg) and polyethylene glycol-polylactic acid (15 mg) were dissolved in chloroform (10 mL) and spin-dried under reduced pressure in a water bath at 37° C. to form a membrane.

Step 2: Deionized water or a buffer solution (a 1×PBS solution, 5 mL) was added to the membrane obtained in step 1 and hydrated at room temperature for 12 h.

Step 3: A micelle solution obtained in Step 2 was passed through a 200-mesh filter membrane to obtain the polymeric micelle preparation of compound 7 (PEG-PLA-ROX with a drug loading rate of 95%).

The micelle could be prepared from different kinds of polymer raw materials, and the desired solvent for the raw materials of the micelle could be selected according to needs, such as DCM, chloroform, tetrahydrofuran, acetonitrile, and acetone. In this test example, chloroform was selected. In step 1, the ratio of the raw materials could be adjusted according to the reagent needs.

Size characterization of the pharmaceutical preparation: The particle size and distribution of the pharmaceutical preparation were determined by dynamic light scattering (DLS). The micelle preparation was assembled in water into nan.oparticles with a dynamic size distribution of 0.121 and an average size of 71.9 nm. The size could be adjusted by polymer composition, preparation method, and the like.

Antitumor Activity Test:

Balb/c mice were subcutaneously injected with 1×10⁶ CT26 tumor cells, and after the tumor grew to about 80 mm³, tail vein injection was conducted every two days (on Day 0, Day 2, and Day 4) for administration. Taking a combination of PEG-PLA-ROX and doxorubicin liposome as an example, there was a blank control group, a doxorubicin liposome group (Doxil, the DOX dosage was 5 mg/kg), a PEG-PLA-ROX+ doxorubicin liposome group (PEG-PLA-ROX+Doxil, the DOX dosage was 5 mg/kg, the ROX dosage was 5 mg/kg), and a PEG-PLA-ROX group (the ROX dosage was 5 mg/kg). After the administration, the mice were observed for another 18 d.

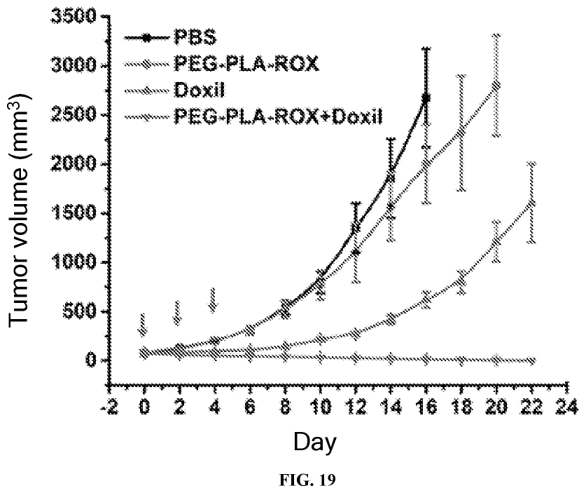

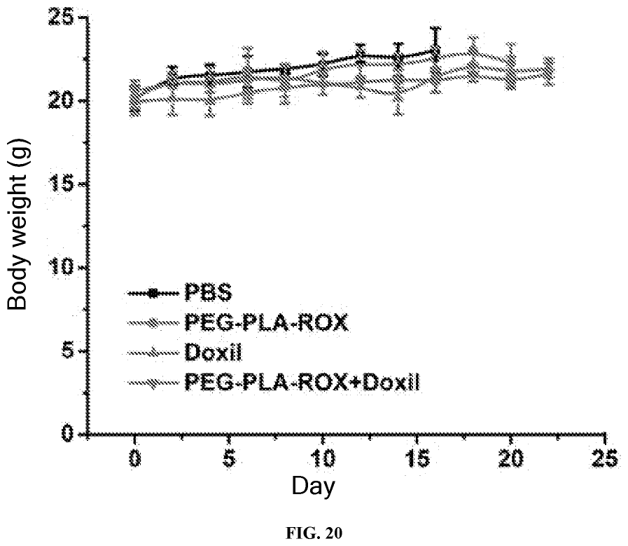

The results are shown in FIG. 19. The combination of the two preparations had an anti-tumor effect significantly better than that of the doxorubicin liposome alone. After 22 d, the tumors in the combination group were eliminated, while the tumors in the doxorubicin liposome group still showed a growth trend. FIG. 20 showed that the body weight of mice in the combination group did not decrease, indicating that the drug had high biosafety and fewer side effects.

After being prepared into a pharmaceutical preparation, the combination of PEG-PLA-ROX with chemotherapeutic drugs showed a significant anticancer activity, which had the greatest therapeutic effect, showing a desirable prospect for use.

What is claimed is:

1. A method of an application of N-(3-hydroxypyridine-2-carbonyl)glycine and derivatives of the N-(3-hydroxypyridine-2-carbonyl)glycine in a preparation of an antitumor drug sensitizer, wherein the N-(3-hydroxyp iridine-2-carbonyl)glycine and the derivatives of the N-(3-hydroxypyridine-2-carbonyl)glycine are 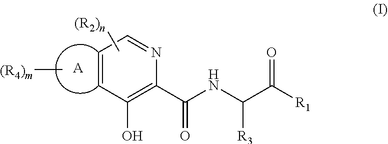
a compound of formula (I)
or a pharmaceutically acceptable salt of the compound, wherein,
R₁ is selected from the group consisting of H, OH, NH₂, a C₁₋₂₀ alkyl, an —O—C₁₋₂₀ alkyl, an —NR—C₁₋₂₀ alkyl, and an —O—C₆₋₁₂ aryl;
R₂ is selected from the group consisting of the H, F, Cl, Br, I, the OH, the NH₂, NO₂, CN, the C₁₋₂₀ alkyl, the —O—C₁₋₂₀ alkyl, the —NH—C₁₋₂₀ alkyl, an C₆₋₁₂ aryl, the —O—C₆₋₁₂ aryl, and a 5- to 10-membered heteroaryl; and the C₁₋₂₀ alkyl, the —O—C₁₋₂₀ alkyl, the —NH—C₁₋₂₀ alkyl, the C₆₋₁₂ aryl, the —O—C₆₋₁₂aryl, and the 5- to 10-membered heteroaryl are optionally substituted by 1 Rₐ, 2 Rₐ, or 3 Rₐ;
R₃ is selected from the group consisting of the H, the F, the Cl, the Br, the I, the OH, the NH₂, the NO₂, the CN, the C₁₋₂₀ alkyl, the —O—C₁₋₂₀ alkyl, the —NH—C₁₋₂₀ alkyl, the C₆₋₁₂ aryl, and the —O—C₆₋₁₂ aryl;
R₄ is selected from the group consisting of the H, the F, the Cl, the Br, the I, the OH, the NH₂, the NO₂, the CN, the C₁₋₂₀ alkyl, the —O—C₁₋₂₀ alkyl, the —NH—C₁₋₂₀ alkyl, the C₆₋₁₂ aryl, the —O—C₆₋₁₂ aryl, and the 5- to 10-membered heteroaryl; and the C₁₋₂₀ alkyl, the—O—C₁₋₂₀ alkyl, the—NH—C₁₋₂₀ alkyl, the C₆₋₁₂aryl, the —O—C₆₋₁₂aryl, and the 5- to 10-membered heteroaryl are optionally substituted by 1 R_{b}, 2 R_{b}, or 3 R_{b};
ring A is a phenyl or absent;
Rₐ is independently selected from the group consisting of the F, the Cl, the Br, the I, the OH, the the NO₂, the CN, a C₁₋₃ alkyl, and a C₁₋₃ alkylphenyl; and the C₁₋₃ alkyl or the C₁₋₃ alkylphenyl is optionally substituted by I halogen, 2 halogens, or 3 halogens;
R_{b} is independently selected from the group consisting of the F, the Cl, the Br, the I, the OH, the NH₂, the NO₂, and the CN;
m is 0, 1, 2, 3, or 4; and
n is 0, 1, or 2.

2. The method according to claim 1, wherein the compound of the formula (1) is selected from the group consisting of:

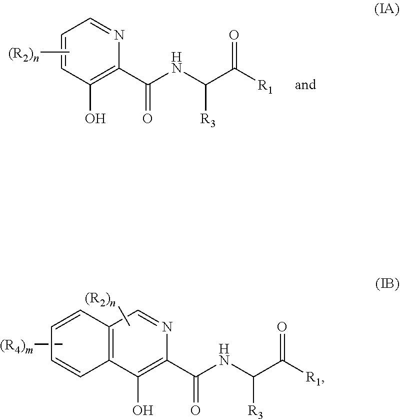

3. The method according to claim 1, wherein the Rₐ is selected from the group consisting of the F, the Cl, the Br, the I, the OH, the NH₂, the NO₂, the CN, and

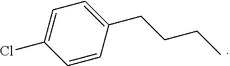

4. The method according to claim 3, wherein the R₂ is selected from the group consisting of the H, the F, the Cl, the Br, the I, the OH, the NH2, the NO₂, the CN, the C₁₋₁₃ alkyl, an —O—C₁₋₃ alkyl, an —NH—C₁₋₃ alkyl, the phenyl, the —O-phenyl or the —O-pyrazolyl, a pyrrolyl, a pyrazolyl, and a triazolyl; and the C₁₋₃ alkyl, the —O—C₁₋₃ alkyl, the —NH—C₁₋₃ alkyl, the phenyl, the —O-phenyl or the —O-pyrazolyl, the pyrrolyl, the pyrazolyl, and the triazolyl are optionally substituted by 1 -Rₐ, 2 Rₐ, or 3 Rₐ.

5. The method according to claim 4, wherein the compound of the formula (I) is selected from the group consisting of the following formulas (1), (2), (3), (4), (5), (6), (7), and (8):

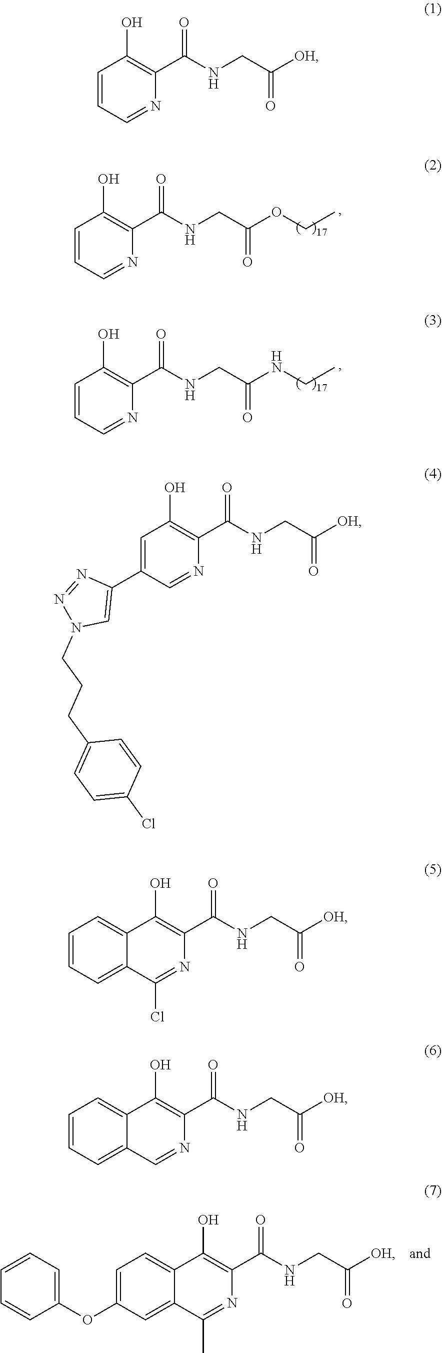

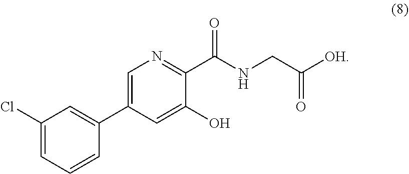 [SMILES: O=C(O)CNC(=O)c1ncc(-c2cccc(Cl)c2)cc1O]

7. The method according to claim 1, wherein the antitumor drug sensitizer is used in a combination with an antitumor drug.

7. The method according to claim 6, wherein the antitumor drug sensitizer and the antitumor drug have a mass ratio of (0.1-20):1.

8. The method according to claim 1, wherein the antitumor drug sensitizer is prepared into a pharmaceutical composition; and the pharmaceutical composition comprises a therapeutically effective amount of the antitumor drug sensitizer and a pharmaceutically acceptable carrier.

9. The method according to claim 1, wherein a method for preparing the antitumor drug sensitizer into a drug liposome composition comprises the following steps:
preparing a liposome membrane comprising dissolving phospholipid or PEGylated phospholipid or a mixture the phospholipid and the PEGviated phospholipid with the antitumor drug sensitizer in a solvent and concentrating at 4° C. to 60° C. to form a membrane; and
performing a hydration comprising adding a deionized water or a buffer solution with an appropriate pH value to the membrane and hydrating at 4° C. to 60° C. for 12 h to 48 h; and
conducting a dialysis on an obtained hydration product in a dialysis bag at a room temperature for 6 to 48 h.

10. The method according to claim 1, wherein a method for loading the antitumor drug sensitizer to prepare a drug micelle composition comprises the following steps:
preparing a micellar membrane comprising dissolving a copolymer and the antitumor drug sensitizer in a solvent and concentrating at 4° C. to 60° C. to form a membrane; and
performing a hydration comprising adding a deionized water or a buffer solution with an appropriate pH value to the membrane, hydrating at 4° C. to 60° C. for 2 h to 48 h, and passing through a filter membrane; wherein the copolymer is selected from the group consisting of a polyvinyl alcohol-polylactide block copolymer and a polyoxyethylene-polyoxypropylene ether block copolymer.

11. The method according to claim 2, wherein the antitumor drug sensitizer is used in a combination with an antitumor drug.

12. The method according to claim 3, wherein the antitumor drug sensitizer is used in a combination with an antitumor drug.

13. The method according to claim 4, wherein the antitumordrug sensitizer is used in a combination with an antitumor drug.

14. The method according to claim 5, wherein the antitumor drug sensitizer is used in a combination with an antitumor drug.

15. The method according to claim 2, wherein the antitumor drug sensitizer is prepared into a pharmaceutical composition; and the pharmaceutical composition comprises a therapeutically effective amount of the antitumor drug sensitizer and a pharmaceutically acceptable carrier.

16. The method according to claim 3, wherein the antitumor drug sensitizer is prepared into a pharmaceutical composition; and the pharmaceutical composition comprises a therapeutically effective amount of the antitumor drug sensitizer and a pharmaceutically acceptable carrier.

17. The method according to claim 4, wherein the antitumor drug sensitizer is prepared into a pharmaceutical composition; and the pharmaceutical composition comprises a therapeutically effective amount of the antitumor drug sensitizer and a pharmaceutically acceptable carrier.

18. The method according to claim 5, wherein the antitumor drug sensitizer is prepared into a pharmaceutical composition; and the pharmaceutical composition comprises a therapeutically effective amount of the antitumor drug sensitizer and a pharmaceutically acceptable carrier.

19. The method according to claim 2, wherein a method for preparing the antitumor drug sensitizer into a drug liposome composition comprises the following steps:
preparing a liposome membrane comprising dissolving phospholipid or PEGylated phospholipid or a mixture of the phospholipid and the PEGylated phospholipid with the antitumor drug sensitizer in a solvent and concentrating at 30° C. to 60° C. to form a membrane; and
performing a hydration comprising adding a deionized water or a first buffer solution with an appropriate pH value to the membrane and hydrating at 4° C. to 60° C. for 12 h to 48 h; and conducting a dialysis on an obtained hydration product in a dialysis bag at a room temperature for 6 h to 48 h.

20. The method according to claim 3, wherein a method for preparing the antitumor drug sensitizer into a drug liposome composition comprises the following steps:
preparing a liposome membrane comprising dissolving phospholipid or PEGylated phospholipid or a mixture of the phospholipid and the PEGylated phospholipid with the antitumor drug sensitizer in a solvent and concentrating at 30° C. to 60° C. to form a membrane; and
performing a hydration comprising adding a deionized water or a first buffer solution with an appropriate pH value to the membrane and hydrating at 4° C. to 60° C. for 12 h to 48 h; and conducting a dialysis on an obtained hydration product in a dialysis bag at a room temperature for 6 h to 48 h.
